(19) United States
(12) Patent Application Publication
(10) Pub. No.: US 2026/0206840 A1
(43) Pub. Date: Jul. 23, 2026

(54) PULMONARY DELIVERY DEVICES

(71) Applicant: Twenty Sixteen (2016) Pharma Limited, Liverpool Merseyside (GB)

(72) Inventors: George Gallagher, Liverpool Merseyside (GB); Anant Pandya, Liverpool Merseyside (GB)

(73) Assignee: Twenty Sixteen (2016) Pharma Limited, Liverpool Merseyside (GB)

(21) Appl. No.: 19/141,507

(22) PCT Filed: Dec. 19, 2023
(86) PCT No.: PCT/EP2023/086641

§ 371 Date: Jun. 20, 2025

(30) Foreign Application Priority Data
Dec. 21, 2022 (GB) 2219330.4

(51) Int. Cl. A24F 40/30; A24F 7/00; A24F 40/10; A24F 40/20; A24F 40/485; A61M 15/06

(52) U.S. Cl. CPC A24F 40/30; A24F 7/00; A24F 40/10; A24F 40/20; A24F 40/485; A61M 15/06; A61M 2206/14

(57) ABSTRACT

A pulmonary delivery device has a first chamber to vaporise a first fluid to form a first vapour, a second chamber 102, which may be provided in a mouthpiece attachable to the device, the second chamber adapted to atomize a second fluid to form a second vapour, and an outlet through which a user can inhale the first and second vapours, the second chamber being in fluid communication with air, the second chamber including a substrate 100 having at least one flavouring or aroma wherein the flavour/aroma is inhaled by drawing air through the second chamber, the second chamber further comprising at least one of an air flow obstruction member 110 and an air flow diversion member to increase uptake of the flavouring or aroma during inhalation of the air.

This invention relates to pulmonary delivery devices, particularly but not exclusively, to pulmonary delivery devices suitable for delivering active molecules and/or medicaments to users, such as nicotine, cannabinoid, peptides, proteins and other lung deliverable medicaments and other vape products.

### TECHNICAL FIELD

Pulmonary delivery devices have widespread uses in modern medicine as they enable drugs and medicaments to be delivered directly to the user's lungs. Moreover, as medicaments delivered to the lungs enter the bloodstream directly, rather than via the body's metabolism, as is the case with oral delivery systems, the benefits of the medicament, especially in pain relief or drug-weaning applications, are felt by the user almost immediately. Another major benefit of pulmonary delivery devices is their ability to deliver drugs without the use of needles.

Existing pulmonary delivery systems take various forms, including inhalator sprays, nebulisers, metered dose inhalers in which the medication is administered in the form of a mist inhaled by the lungs and vapour delivery systems whereby the medicament is admixed to an inhalable vapour (often a water-containing vapour).

A vapour type pulmonary delivery system comprises a carrier liquid, often water or a water-glycol mixture (the glycol serving to stabilise the water droplets when in the vapour form) to which is admixed a desired medicament. The carrier liquid can be vaporised in various ways, such as by spraying it through a nozzle, but in many cases, it is simply heated to form a vapour comprising the carrier liquid and the desired medicament. The resultant vapour is then inhaled by a user to deliver the medicament. However, heating of the medicament can result in undesirable by-products being formed and thus, inhaled by the user. This may also reduce the accuracy of the dose of medicament inhaled.

An example of a vapour type pulmonary delivery system is an e-cigarette that vapes nicotine for inhalation by the user. A nicotine solution (“e-liquid”) is provided in a reservoir (often in the form of a detachable cartridge) and passes along a wick to a heating element where it is vaporised and can be inhaled by the user. Generally, a length of resistance wire connected to a power source, such as a battery, is coiled around the wick. When activated the wire heats up, turning the e-liquid to vapour which is then inhaled by the user. Such a device has clear advantages over smoking conventional cigarettes since far fewer, and safer, ingredients are inhaled by the user than when smoking an ordinary tobacco cigarette. However, regular users have noted that the e-cigarettes do not “hit the spot” in the manner of a conventional tobacco cigarette. This is due to the wet vapour quickly condensing in the mouth of the user, resulting in the majority of nicotine absorption being through the mucous membranes of the nose, throat and airway leading to the lungs. In contrast, with conventional cigarette smoking, nicotine passes straight into the lungs giving a rapid absorption into the blood stream and a corresponding “quick hit”.

An alternative type of e-cigarette is an atomizer inhaler. This type of device provides a better, more rapid absorption of nicotine due to the use of a cold, pressurized vapour in place of a heated vapour, thereby avoiding significant condensation of the vapour in the mucosa of the nose and throat. However, the cold and dry sensation provided by this type of device results in the overall experience differing greatly to smoking conventional cigarettes resulting in such devices being less popular than the vaping type e-cigarettes, resulting in none or poor compliance.

The Applicant's earlier PCT Patent Publication No. WO 2015/079197 provides a solution to this problem by the provision of a first chamber having a heat source adapted to thermally vaporise a quantity of a carrier liquid to form a heated first vapour and a second chamber adapted to atomize a quantity of a second liquid containing an active molecule or medicament without heating of the second liquid to form a mist of a second vapour having a lower temperature than the first vapour, and an outlet via which, in use, a user can inhale a mixture of the first and second vapours. In this manner, rapid absorption of an active molecule contained in the second vapour is achieved while providing the user with a desirable heat sensation on inhalation provided by the vapour of the “warm” first liquid.

Electronic nicotine delivery systems (ENDS) are often provided to deliver flavours in conjunction with the vaporised nicotine. However, a problem exists due to the risk of inhalation of the ingredients of the flavourings, such as oils, with some being potentially carcinogenic when vaporised or delivered to the lung. Furthermore, it is difficult to change the flavour of the system without carryover or contamination in the main body of the system. Coffee, tobacco, mint and fruit flavours are often difficult to alternate due to their characteristic aromas. Currently, the flavours form an integral part of the propylene glycol/water carrier liquid which contaminates the main body of the device.

The Applicant's earlier Publication No. WO 2019/030602 A1 provides a mouthpiece for a pulmonary delivery device having a separate chamber for delivery of the flavour, the chamber having its own air intake, thus ensuring that the flavour is not in contact with the heat source of the device. This not only prevents contamination of the main device but also prevents degradation of the flavour. The device has also been proven to increase the amount and quality of the flavouring delivered to the user. However, it is desirable to improve the device further to maximise the amount of flavour delivered to the user.

It is an aim of the present invention to provide an improved pulmonary delivery device and/or a mouthpiece for the device that maximises flavour delivery to the end user.

### SUMMARY OF THE INVENTION

Accordingly, a first aspect of the present invention provides a pulmonary delivery device comprising: a first chamber adapted to vaporise a quantity of a first fluid to form a first vapour; a second chamber adapted to atomize a quantity of a second fluid to form a second vapour, and an outlet via which, in use, a user can inhale a mixture of the first and second vapours wherein the second chamber comprises a passive atomiser wherein the second chamber is selectively or continuously in fluid communication with air, the second chamber including a substrate having at least one flavouring or aroma wherein the flavour/aroma is inhaled by drawing air through the second chamber, the second chamber further comprising at least one of an air flow obstruction member and an air flow diversion member to increase uptake of the flavouring or aroma during inhalation of the air.

The incorporation of one or multiple air flow obstruction members or air flow diversion members serves to optimize air velocity through the chamber, increase contact of air with a larger surface area of the substrate and/or recycle air through the chamber to provide a greater uptake of the flavouring in the substrate during inhalation.

The at least one air flow obstruction member is preferably provided upstream of the substrate in the second chamber. This creates turbulence in the air flow prior to entry to the substrate to slow down air flow through the substrate and increase favour uptake. Preferably, the first chamber and the second chamber each have at least one air inlet and the device is provided with at least one outlet, preferably a single outlet. In the context of this disclosure, the term ‘upstream’ means towards the air inlet and ‘downstream’ means towards the outlet.

The at least one air flow obstruction member may comprise a baffle extending at least partially across the chamber, preferably having at least one air passage therethrough in the form of a hole. More preferably, the baffle comprises a disc having a plurality of bore holes therethrough. Multiple air flow obstruction members may be provided in the chamber to adjust air flow through the substrate. For example, multiple discs with bore holes may be provided in the chamber, preferably wherein the bore holes of adjacent discs are provided out of phase. The chamber may be provided with multiple substrates, for example being sandwiched between air flow obstruction members.

Alternatively or additionally, the second chamber of the mouthpiece is configured to direct air flow through as much of the substrate as possible, thereby increasing flavour uptake and suction resistance. In one embodiment, the substrate may be housed within a cage suspended within the chamber, the cage having an end cap at the upstream end of the device and having an outlet at the downstream end. The sides of the cage may be provided with multiple spaced apart apertures which serve as entry points for air flow into the substrate. In this manner, air overshoots the initial part of the substrate due to the end cap and enters via the multiple inlet apertures, thereby directing air through as much of the substrate as possible.

Additionally, a baffle or vane may be provided near to the start of the substrate downstream of the end cap to further increase air flow into the substrate. Additionally (or alternatively), a downstream end cap with at least one aperture, preferably being substantially central in the cap, may be provided at the outlet end of the substrate.

It is to be appreciated that the mouthpiece or pulmonary delivery device may be provided with a combination of the aforementioned features, such as both a cage and disc combination, to enhance flavour absorption and/or suction resistance.

Alternatively, or additionally, the at least one air diversion member may comprise at least one subsidiary channel or conduit extending around the part of the chamber that houses the substrate to enable air to be recycled through the substrate, thereby enhancing air saturation with flavour and increasing suction resistance. Preferably, one or more side or subsidiary conduits of a narrower cross-sectional diameter than the chamber extend from a junction off the chamber prior to the substrate to a junction beyond the substrate. This enables a proportion of the air that has travelled through the substrate to be diverted back along the subsidiary channel and be re-cycled through the substrate.

In one embodiment, multiple subsidiary conduits are provided extending from the chamber at a junction prior to the substrate to a junction beyond the substrate. The chamber and subsidiary conduits are configured to ensure air is recycled back through the substrate rather than causing air to bypass the substrate. This may also be achieved or enhanced by optimizing the cross-sectional area of the subsidiary channels relative to the cross-sectional area of the outlet of the chamber to ensure air velocity is sufficient to prevent bypassing of the chamber. Alternatively, air flow obstruction and/or diversion members may be provided at the entry and exits to the subsidiary conduits to encourage air flow through the downstream inlets to the subsidiary channels while obstructing flow from the chamber into the upstream inlets to the subsidiary channels.

In one embodiment, at least one disc with apertures is provided in the chamber at the point where the downstream junctions meet the chamber and a funnel and duct is provided at the point where the upstream junctions meet the chamber. The disc aids the diversion of flow into the subsidiary channels while the funnel and duct obstructs flow into these channels, directing flow through the substrate.

In a preferred embodiment of the present invention, the first chamber is adapted to vaporise a quantity of a first fluid to form a relatively warm, wet first vapour, for example by providing the first chamber with a heat source and the second chamber is adapted to atomize a quantity of a second fluid to form a relatively cold second vapour without any heating. Additionally or alternatively, both chambers may be provided with a heat source. Preferably, the heat source to one or each chamber may be switched on and off.

It is to be appreciated that any of the embodiments discussed could be incorporated into a second chamber of a mouthpiece having first and second chambers that is connectable to a main body portion of a pulmonary delivery device, the main body portion optionally having a heat source, such as a heating element. The first chamber is preferably provided with, or connected to, a heat source for vaporisation of the first fluid thereby creating a first “warm” vapour.

To this end, a second aspect of the present invention provides a mouthpiece for a pulmonary delivery apparatus, the mouthpiece having a first inlet end and a second outlet end and comprising: a first mouthpiece chamber adapted for receipt within a main body of a pulmonary delivery device at the inlet end of the mouthpiece and a second mouthpiece chamber, the second chamber being adapted for receipt of at least one substrate carrying at least one flavouring or aroma, the second chamber having at least one air inlet at the inlet end whereby the second chamber is selectively or continuously in fluid communication with air to enable inhalation of the flavour/aroma by drawing air through the second chamber, the second chamber further comprising at least one of an air flow obstruction member and an air flow diversion member to increase uptake of the flavouring or aroma during inhalation of the air.

The substrate containing the flavouring or aroma is preferably in the form of a foam. The foam may be polyurethane, polyethylene or other suitable medical grade composition of suitable porosity to deliver optimal flavour. The foam can be placed in a chamber for forming the second chamber of a mouthpiece or the pulmonary delivery device. However, other porous materials may serve as the substrate.

The flavourings and/or aroma may be selected from essential oils of dried flowers, buds, leaves, stems, fruit, seeds, peel, bark, or root e g oil of peppermint, spearmint, eucalyptus, wintergreen, clove, cardamom, cinnamon, bitter almond, coriander, caraway, ginger, juniper, orange, bitter orange, lemon, grapefruit, bergamot, thyme, fennel rosemary etc., natural flavors and aroma agents of essential oils or concentrates of flavor components with natural origin from e g fruits, berries, nuts, spices, mints, tobacco, cocoa, coffee, tea, vanilla, liquorice, caramel, toffee, honey, wine, liquors and brews, synthetic flavors and aroma agents consisting of mixtures of chemicals comprising hydrocarbons, alcohols, aldehydes, esters, ketones, ethers and oxides blended to match the natural flavor of e g fruits, berries, nuts, spices, mints, tobacco, cocoa, coffee, tea, vanilla, liquorice, caramel, toffee, honey, wine, liquors or brews and mixtures thereof.

The flavours may also be configured to mimic the flavour of different foods and/or drinks. For example, sweet and savoury flavours. Such flavours are particularly beneficial for use during enteral feeding. Enteral feeding delivers food directly to the stomach and intestine and hence flavour is lost. This way the patient/subject could be provided with a device of the present invention to obtain increased satisfaction during enteral feeding with delivery of flavour to the mouth. This may be further enhanced by delivery of a warm or cold vapour depending upon whether the food is normally served hot or cold. With the heated option, wherein a heat source is activated in one of the chambers, the flavour will be accompanied by a warm feel in the mouth to mimic eating warm food and with a non-heated option, wherein the heat source is switched off, just flavour will be delivered for mimicking cold foods.

The first fluid may comprise a carrier liquid (i.e. a liquid capable of forming a stable vapour), which may be an inert (non-medicated carrier liquid), such as water, or a water-glycol mixture. An active ingredient such as nicotine may be included with the first fluid. Alternatively, this may be included in the second chamber.

Preferably, the flavour is provided in a solid or semi-solid form within the second chamber in a suitable formulation. Optionally, the volume of air inhaled through the second chamber may be selectively adjustable thereby enabling the amount of flavour inhaled to be varied and deposited in the mouth to enhance the flavour and avoid thermal decomposition thus avoiding carcinogenic degradant products.

Preferably, the mouthpiece according to the second aspect forms an outlet for a pulmonary delivery device, the mouthpiece preferably being reversibly detachable. In this manner, the flavour may be provided in the mouthpiece to enable its easy replacement, for example to change to a different flavour, such as a different flavour of food, or to allow replenishment of the flavour. The second chamber of the mouthpiece preferably at least partially surrounds the first chamber, the second chamber having at least one air inlet and the flavour being provided in the second chamber. In one embodiment, the second chamber comprises multiple chambers extending along each side of the first chamber. In another embodiment, the mouthpiece comprises a central first mouthpiece chamber and a concentric outer second mouthpiece chamber, the second chamber having at least one air inlet.

It is to be appreciated that the mouthpiece could be permanently attached to a pulmonary delivery device, for example forming part of the device but more preferably, the mouthpiece is a separate component.

The pulmonary delivery device may include only the first chamber with the mouthpiece providing the second chamber, preferably the mouthpiece providing an extension to the first chamber in addition to providing the second chamber that preferably surrounds the extension to the first chamber.

Preferably, the first chamber of the mouthpiece is dimensioned for receipt within a main first chamber provided in a pulmonary delivery device that preferably produces heated or warm vapour. Suitable attachment means is provided for attachment of the mouthpiece to the main device. For example, the end of the first mouthpiece chamber may be threaded or comprise a snap-fit arrangement. The outer second mouthpiece chamber may terminate in a flange having at least one air inlet wherein the flange is received on a top surface of the main first chamber of the main body of the pulmonary delivery device.

Alternatively, an annular flange may extend laterally from a region of the first mouthpiece chamber that contacts that the main body of the device, the flange being provided with holes to allow air into the second mouthpiece chamber surrounding the first chamber.

The entry of air through the inlets may be controllable and/or variable either manually or automatically. For example, the number of air inlets that are open may be adjustable to suit the preference of the user. Any suitable mechanism may be employed to allow opening and closing of one or more of the multiple inlets.

It is to be appreciated that it is possible to control the composition of the mixture by controlling the quantity of vapour released from one or each of the main chambers or mouthpiece chambers. Suitably, the delivery apparatus may comprise a controller adapted to control, in use, the composition of the first and/or second vapours in the mixture, that is to say, by controlling the relative amounts of the first and second vapours in the mixture, or the ratio between the two. The controller could be adapted to switch one or the other of the vaporisers on or off manually or automatically, thus providing the option of delivering one or the other of the liquids in vapour form. Automatic control may be via an externally controllable device, such as smart phone, computer or the like.

It is to be appreciated that in embodiments where a heat source is provided in the first chamber, the formation of a relatively warm, wet first vapour is provided by the first chamber being adapted to thermally vaporise a quantity of a first fluid to form a vapour of a higher temperature than the vapour formed by the second chamber, for example by providing the first chamber with a heat source. The second chamber has no heat source and therefore provides a relatively cooler second vapour.

The first main chamber of the pulmonary delivery device preferably comprises a vaporiser in the form of an electric heater, for example a battery-powered resistive heating wire or coil. The current delivered to the resistive heating wire or coil can be used to control the temperature of the wire or coil, and thus regulate and/or control the heating and vaporisation of the liquids. In an embodiment of the invention, the heater comprises a hydrophilic or super-hydrophilic foil, which coats with a film of the liquid to be vaporised. A current can be passed through the coil to heat it, thereby vaporising the liquid. Alternatively, a ceramic heater may be used as the heat source. The use of a ceramic or other suitable material heater may be preferred as it reduces the potential for metals to be transferred/inhaled into the user's lung i.e. metal elements exposed to the high temperature may result in harmful metal residue being delivered to the lungs.

A feedback circuit may be provided to thermostatically regulate the temperature, or temperature profile of the heater. For example, a circuit may be provided to monitor the resistance of the wire or coil (the resistance being dependent on the wire or coil's temperature) and to adjust the current in the wire or coil such that the resistance, and hence the temperature, is controlled.

Preferably, the heat source may by switched on or off.

The vaporiser suitable for use in the first chamber of a pulmonary delivery device according to the present invention may comprise an electric heater adapted to vaporise a quantity of vaporisable liquid in contact therewith, the vaporiser further comprising a circuit configured to apply a time-dependent heating and/or cooling profile by temporally controlling an electric current in the heater in response to a measured temperature thereof.

Such a configuration, that is to say, a time-dependent heating and/or cooling profile, suitably controls the vaporisation of the liquid or liquids more precisely and reproducibly, and/or improves the longevity of the heater, which is suitably a heating wire, foil, coil or ceramic tube.

Other heating devices could equally be used, such as thermionic emitters, Peltier devices, infrared emitters and so forth, and the invention is not restricted to resistive heater wires, foils or coils.

Preferably, the particles produced using suitable formulation in the mist provided by the second chamber have an average diameter of 5 to 50 μm, more preferably 8-35 μm. The flavour is provided in a suitable formulation to provide the desired particle size. For example, a suitable formulation may include viscosity enhancers, surfactants, stabilizers and/or humectants to optimise the organoleptic properties.

In an embodiment of the invention, for example, a nicotine weaning device, the pulmonary delivery system resembles a cigarette, a pipe or a cigar. In such a situation, the first fluid can comprise an inert mixture of water and glycol and the second fluid can contain a mixture of a propellant, flavouring and/or the desired medicament, in this case, liquid nicotine. The device can thus be programmed to deliver a certain dose of medicament (nicotine) in each “puff” of the device, or over a given period, such as a day. However, the nicotine may also be provided in the first fluid, as desired and, indeed, in certain cases, this may be preferred with only the flavouring being contained in the second fluid expelled from the second chamber.

In one embodiment, the vaporiser of the first main chamber of the pulmonary delivery device suitably comprises a reservoir for retaining, in use, a quantity of the respective liquid and a conveyor adapted to convey, in use, the liquid from the reservoir to a heater. In an embodiment of the invention, the reservoir comprises a vial and the conveyor comprises a wick extending between the interior of the vial and the heater. Suitably, a resistive heating wire, such as that described herein, can be wrapped or coiled around the wick to vaporise the liquid. The conveyor may comprise a capillary tube extending between the vial and the heater.

The first and/or second fluids may suitably comprise a solvent and a stabiliser in an appropriately designed formulation/recipe. The stabiliser is suitably adapted to stabilise droplets of the solvent in air. The carrier liquid can comprise any one of more of the group comprising:
solubilizers, solvents and mixtures thereof such as water, alcohols such as glycerol, propylene glycol, polyethylene glycol, vegetable oils, mineral oils, lipids, cyclodextrins etc. Surface active agents such as anionic agents with carboxylate ions, sulphate groups and sulphonate groups; cationic surfactants, nonionic surfactants such as polyol ethers, polyoxyethylene esters and ethers, poloxamers; amphoteric surfactants, natural emulsifiers, sucrose esters and alkylpolyglucosides;
antioxidants such as ascorbic acid and its salts and derivatives tocopherols (vitamin E), thiol derivatives such as cysteine and acetyl cysteine, butylated hydroxyanisole (BHA), butylated hydroxytoluene (BHT), sodium hydrogen sulfite, sodium metabisulfite, sodium thiosulfate;
absorption enhancers such as alcohols, azone;
chelating agents, such as EDTA and galates;
minerals, such as fluorides;
propellants, such hydrofluroalkanes (HFA) Chlorofluorocarbons (CFCs), carbon dioxide etc.; sweeteners such as artificial sweeteners e g saccharin and its sodium and calcium salts, aspartame, acesulfame and its potassium salt, thaumatin and glycyrrhizin, polyhydric alcohols such as sorbitol, xylitol, mannitol and glycerol, glucose, fructose, galactose, sucrose, lactose, maltose and mixtures thereof;
and
pH regulators and buffering agents such as sodium, potassium or calcium hydroxide; bicarbonates, citrates and phosphates, etc.

One or both of the first or second fluids suitably comprise an active molecule or medicament. The active molecule, excipient or medicament may comprise any one or more of the pharmacologically active compounds from the group comprising:
Peptides and proteins;
H2-receptor antagonists such as cimetidine; and ranitidine;
Prostaglandin analogues such as misoprosol;
Proton pump inhibitors such as lansoprazole; omeprazole; and pantaprazole;
Agents to treat food allergies such as sodium cromoglicate;
Cardiac glycosides such as digoxin;
Diuretics such as amiloride; bendroflumethizide; indapamide; furosemide; hydrochlorothiazide; and xipamide;
Drugs for arrythmias such as procainamide; lidocaine; propranolol; atenolol; bisoprolol; carvedilol; pindolol; and nebivolol;
Antihypertensives and agents for treatment of angina such as clizapril; lisinopril; ramipril; trandolapril; amlodipine losartan; glyceryl trinitrate, isosorbide mononitrate; amlodipine; diltiazem; felodipine; isradipine; lacidipine etc.;
Lipid regulating drugs such as statins;
Drugs acting on the respiratory system such as salbutamol; terbutaline; bambuterol;
Antihistamines such as cinnareizine; promethazine; perphenazine and prochlorperazine;
Hypnotics such as zolpidem; zopiclone; clomethiazole;
Anxiolytics such as benzodiazepines; buspirone;
Antipsychotic agents such as benperidol; fluphenazine; pimozide and amisulpride;
Antidepressant drugs such as tricyclics; mianserin; MAOIs; SRIs; reboxetine etc.;
Central nervous system (CNS) stimulants such as methylphenidate;
Drugs used in the treatment of nausea such as antihistamines; domperidone; metoclopramide; 5HT3 antagonists; hyoscine, and betahistine;
Opioid analgesics such as morphine; buprenorphine and fentanyl;
Anti-migraine drugs such as 5HT1 agonist and ergot alkaloids;
Drugs used in treatment of Parkinsonism such as apomorphine; bromocriptine; lisuride; haloperidol and ergot alkaloids;
Drugs used in substance dependence such as nicotine and buprenorphine;
Drugs used in dementia such as rivastigmine; dihydroergotamine; dihydroergocristine and dihydroergocryptine;
Antibiotics; antifungals; antivirals and antimalarials;
Drugs used in treatment of diabetes;
Glucocorticoid therapy using steroids such as betamathasone and dexamethasone;
Male and/or female sex hormones such as estradiol; norethisterone; progesterone; testosterone and esters;
Pituitary hormones such as vasopressin and desmopressin;
Drugs affecting bone metabolism such as calcitonin and bisphosphonates;
Endocrine drugs such as bromocriptine and cabergoline;
Contraceptives such as oestrogens; progestrogens and combinations thereof;
Drugs used in urinary frequency and enuresis such as oxybutynin and desmopressin;
Drugs used in erectile dysfunction such as apomorphine and sildenafil;
Drugs used in malignant disease and immunosuppresion such as buslfan; antimetabolites; alkaloids; corticosteriods; hormones and interferons;
Non-steroidal anti-inflammatory drugs such as diclofenac; piroxicam and refoxicab;
Drugs used in treatment of gout such as colchicine;
Drugs used in neuromuscular disorders such as neostigmine and pyridostigmine;
Muscle relaxants such as diazepam; tizanidine;
Vaccines delivered by subcutaneous route;
Agents for the treatment drug withdrawal such as nicotine withdrawal symptoms; and Cannabinoids.

At least one active compound may be a nutraceutically active compound. A “nutraceutically active compound” is a compound, derived from a natural origin (animal or vegetable) that has a beneficial and/or therapeutic effect on the human or animal body in the treatment of a condition. Such compounds may be regarded as nutrients.

Suitable nutraceutically active compounds may be natural products extracted from animals or vegetables. Examples of suitable nutraceutically active compounds include:
Carotenoids such as lycopene, lutein, astaxanthin and □-carotene; glucosamine or Nacylglucosamine; ubiquinone;
Vitamins such as vitamins A, C, D and E; Rosmarinic acid; Honokiol; Magnolol; Chlorogenic acid; Oleuropein; Methylsulphonylmethane (“MSM”); Collagen and Chondroitin; Boswellin and boswellic acid; Escin and esculin; Tumeric extracts such as curcuminoids and etrahydrocurcuminoids; Gingerol and gingerone; Triterpenes such as ursolic acid and oleanolic acid; Diterpenes such as asiaticoside, sericoside and ruscogenins; Hydroxycitric acid (“HCA”) and niacinamide hydroxycitrate; Trigonellin; and Corosolic acid; Saw palmetto; and St John's Wort.

The device suitably comprises a battery, such as a disposable or rechargeable battery, for powering the heater of the first chamber and/or control circuitry.

The heater is suitably switched on or off using a switch. The switch is suitably an automatic switch that is triggered by the user inhaling on the device. The switch may therefore comprise a pressure-activated switch associated with the outlet of the device, whereby when a user draws on the device, the switch is turned on thereby automatically switching on the heater, and whereby the heater is switched off again when the user ceases drawing on the device. Suitably, the device additionally comprises a second pressure-sensitive switch for monitoring the pressure of the ambient air.

### BRIEF DESCRIPTION OF THE DRAWINGS

Embodiments of the invention shall now be described, by way of example only, with reference to the accompanying drawings in which:

FIG. 1 is a perspective view of a pulmonary delivery device according to the prior art;

FIG. 2 is an exploded view of the pulmonary delivery device of FIG. 1;

FIG. 3A is a cross-section through a mouthpiece according to the prior art for attachment to a pulmonary delivery device;

FIG. 3B is a perspective view of the mouthpiece shown in FIG. 3A with a flavour block fully inserted into the second chamber;

FIG. 3C is a perspective view of the mouthpiece shown in FIG. 3A with a flavour block partially inserted into the second chamber;

FIG. 3D is a perspective view of the mouthpiece of FIG. 3A illustrating the air inlets of the second chamber;

FIG. 4 is perspective view of a prior art pulmonary delivery device and mouthpiece;

FIG. 5 illustrates air flow velocity through a flavour substrate/block provided in a chamber;

FIG. 6A is a schematic diagram of a second chamber for a mouthpiece according to one embodiment of the present invention;

FIG. 6B illustrates air flow velocity through the second chamber shown in FIG. 6A;

FIG. 6C is an expanded view of FIG. 6C in the region of the flavour block;

FIG. 7A is a schematic diagram of a second chamber for a mouthpiece according to another embodiment of the present invention;

FIG. 7B illustrates the second chamber shown in FIG. 7A with flavour blocks removed;

FIG. 7C illustrates air flow velocity through the second chamber shown in FIG. 7A;

FIG. 7D is an expanded view of FIG. 7C in the region of the flavour blocks;

FIG. 8A is a schematic diagram of a second chamber for a mouthpiece according to yet another embodiment of the present invention, with a side section of the chamber wall removed;

FIG. 8B is an expanded cross-sectional view of part of the second chamber shown in FIG. 8A;

FIG. 8C illustrates air flow velocity through the second chamber shown in FIG. 8B;

FIG. 9A is a schematic diagram of a second chamber for a mouthpiece according to yet a further embodiment of the present invention, with a side section of the chamber wall removed;

FIG. 9B is an expanded cross-sectional view of part of the second chamber shown in FIG. 9A;

FIG. 9C illustrates air flow velocity through the second chamber shown in FIG. 9B; and

FIG. 10 is a schematic diagram of a second chamber for a mouthpiece according to another embodiment of the present invention;

### DETAILED DESCRIPTION

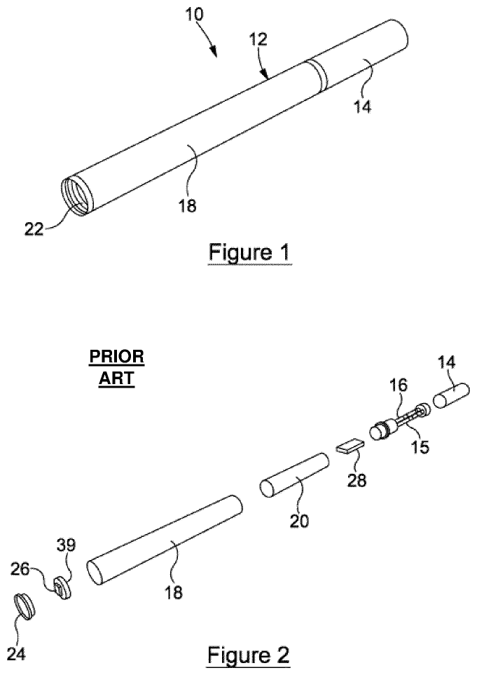

Referring to FIGS. 1 and 2 of the accompanying drawings, a prior art pulmonary delivery device 10 comprises a generally cylindrical main body portion 12 optionally adapted to resemble a cigarette. The main body portion 12 comprises a tubular filter chamber 14 encasing dual vaporiser chambers 15, 16 and a tubular battery chamber 18 encasing a rechargeable battery 20. The tip 22 of the main body 12 is closed off by a translucent end cap 24, behind which sits an LED indicator light 26 that illuminates when the device 10 is in use. A control circuit 28 is contained within the body 12, which comprises a programmable circuit for controlling the operation of the device 10, in use.

The device 10 comprises a first pressure sensor (not visible) located within the filter chamber 14, which has an outlet aperture therein through which, in use, vapour generated by the device 10 can be inhaled by a user. When the user draws on the filter chamber 14, air is able to enter the device through at least one inlet and the pressure sensor (not visible) activates the first and/or second vaporisers 15, 16 to form a mixed vapour comprising the first and/or second liquids, to be inhaled.

The dual vaporiser chambers 15, 16 comprise a pair of separate reservoirs containing first and second liquids respectively. In the illustrated embodiment one of the chambers has a heat source and the other does not. For example, a first reservoir 15 contains a first liquid and comprises a capillary wick, which absorbs the liquid, and whose end touches a heater element in the form of a pyramid-shaped, super-hydrophilic foil, which is wetted by the first liquid, in use. The heater element could alternatively comprise a resistive heating coil, which is wrapped around the wick. In any event, the heater element is connected to the battery 20 under the control of the control circuit 28.

The second reservoir 16 contains a second liquid that is held under pressure within the reservoir and includes a pressure release valve or flow control valve (not shown). When the user draws on the filter chamber, the pressure sensor activates the valve to propel the second liquid out of the second reservoir as a fine mist or vapour. The absence of any heating element results in a cold vapour being released from the second reservoir.

Thus, when the heater is switched on, the first chamber acts as a “warm vapour chamber” with the first liquid being evaporated and forming a warm vapour within the interior of the filter chamber 14. Simultaneously, a relatively cold vapour A is released into the interior of the chamber 14 from the second reservoir (the “cold vapour chamber”) thus allowing the warm and cold vapours to mix in the hollow space of the filter chamber, before being inhaled by the user, via the outlet aperture of the device.

The first liquid may comprise, for example, a mixture of glycerol, propylene glycol and water and the second liquid may comprise nicotine and a suitable propellant. Preferably, the particles forming the mist of the second liquid are less than 10 μm in diameter, more preferably less than 5 μm. In this manner, nicotine (or other active molecule provided in the second liquid) is delivered deep into the lungs to allow for its quick absorption into the bloodstream via the lungs. However, the simultaneous delivery of a warm, wet vapour in the form of the vaporised first liquid provides the user with a sensation that more closely resembles that experienced during the smoking of a conventional tobacco cigarette. The active molecule is not in direct contact with the heater element, reducing the potential for its thermal degradation which may have resulted in the user inhaling harmful by-products. In contrast, only glycerol and water are in contact with the heater element which do not result in the production of harmful by-products upon their thermal degradation.

The device may also be provided with a suitable control circuit 28 that may control the delivery of the first and/or second vapours from their respective chambers. The ability to deliver nicotine from a pressurized chamber without heating allows for more accurate nicotine dosing using the device of the present invention than the delivery of nicotine using the heated vapour method. It is to be appreciated that the delivery of the wet warm vapour and the cold vapour may be controlled and the content of the mixed vapour may be adjusted as required.

The afore-mentioned prior art device provides many potential advantages over earlier pulmonary delivery devices. The active ingredient, such as nicotine or a cannabinoid, is inhaled as small particles (<10 μm) resulting in it being delivered deep into the lungs of a user enabling its fast absorption into the bloodstream. The simultaneous delivery of a warm inert vapour enhances the flavour and sensation of the inhalation. The active ingredient is not subject to thermal degradation, leading to a reduction in any harmful by-products and increasing the accuracy and reproducibility of the dosage.

However, a problem arose with the delivery of flavours, such as oils or other extracts, in the hot wet vapour which led to contamination of the device and degradation of the flavour. A mouthpiece, which may be formed integrally or be detachable from the rest of the device, was developed which housed the flavour and had its own air inlet, thereby avoiding degradation of the flavour and preventing contamination of the rest of the device. This device provides delivery of the flavor or aroma in a “cold” atomized vapour. This ensures that the flavour is delivered to the mouth and prevents degradation of the flavour to harmful by-products that may occur on heating. It also enables the flavour of an ENDS to be changed without contamination or carryover into the tank system. Coffee, tobacco, mint and fruit flavours are often difficult to alternate due to their characteristic aromas. The device enables the use of a flavour block in the aerosolising chamber to reduce or eliminate cross-contamination of flavour from use to use with simple changeover.

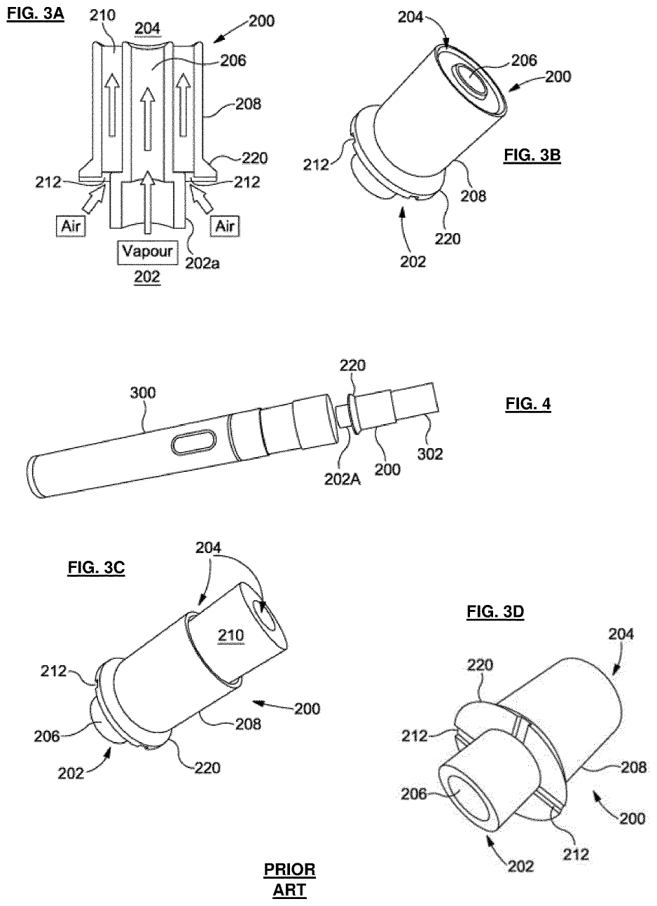

An example of the prior art mouthpiece is shown in FIGS. 3A-3D and FIG. 4. Mouthpiece 200 attached to a conventional pulmonary delivery device, such as the device 300 shown in FIG. 4. However, it is to be appreciated that the mouthpiece may form an integral part of the whole device. The mouthpiece has a first inlet end 202 and a second outlet end 204 with a first central chamber 206 consisting of a cylindrical tube which is adapted for receipt within a main body of a pulmonary delivery device 300 at the inlet end 202 of the mouthpiece. A second chamber 208 concentrically surrounds the first chamber, the second chamber being adapted for receipt of at least one flavour or aroma 210 and having at least one air inlet 212 at the inlet end.

The first chamber 206 of the mouthpiece extends beyond the inlet end of the second chamber. The outlet ends 204 of the chambers are substantially co-terminus. The inlet end of the second chamber is provided with an annular flange 220 extending perpendicularly from the second chamber, the flange having the at least one air inlet 212 in fluid communication with the second chamber. A portion 202a of the first chamber extends beyond the flange for receipt within a main body of a pulmonary delivery device and the flange is adapted to rest or engage with the sides of the main body, whereby the air inlets of the second chamber are positioned beyond the sides of the main body. A filter tip 302 may also be received at the outlet end 204 of the mouthpiece (see FIG. 4).

Any desired flavour block 210 is provided within the second chamber 208. In this manner, when the mouthpiece 200 is attached to the pulmonary delivery device and a user inhales with the mouthpiece, warm wet vapour from the main body of the device enters the first chamber 206 of the mouthpiece. Simultaneously, air is able to enter the second chamber through air inlets 212 to atomise the flavour in the flavour block. This results in the user having a mixture of warm wet vapour with a cold vapour that contains the flavour, addressing the cold sensation of delivery of some nicotine products or other active agents without the potential of harmful ingredients of flavourings being orally deposited.

This arrangement is not only less detrimental to health because the flavouring, such as an oil, is not subjected to heat, but also enables the mouthpiece to be easily changed for a mouthpiece having a different flavor block, without any contamination of the main pulmonary delivery device.

While fit for purpose, it is desirable to improve the amount of flavour provided to the user with each inhalation and also to increase the suction resistance with each puff as this enhances the smoking experience for the user. The present invention addresses these issues by the incorporation of one or multiple obstructions/restrictions and/or diversion members to alter the air flow prior to its entry into the flavour substrate/block. Additionally, or alternatively, the air may be recycled for multiple journeys through the flavour block prior to inhalation. The obstructions/restrictions serve to create turbulence in the air flow prior to entry to slow down air flow through block, presenting air to a greater surface area of the flavour substrate and/or recirculating air through the flavour substrate to maximise air saturation.

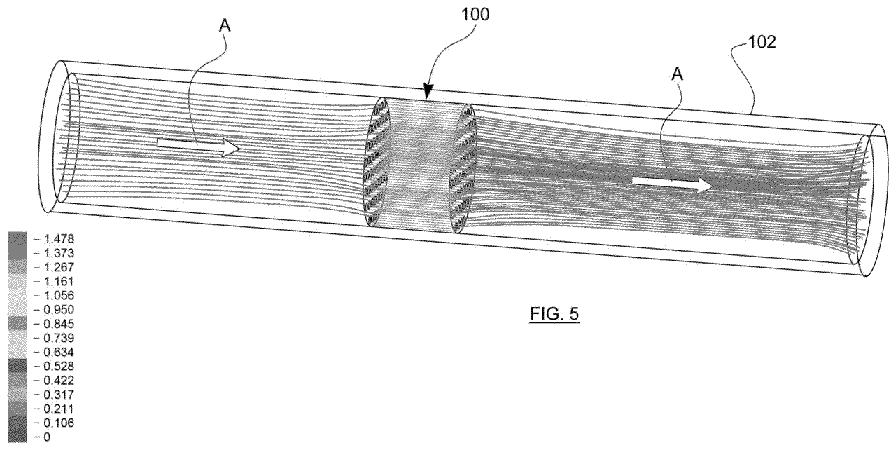

Investigations were conducted to determine the effect of air flow on flavour uptake and draw resistance through a channel provided with a substrate. FIG. 5 of the accompanying drawings illustrates air flow A through a flavour substrate 100, such as a foam sponge containing flavouring, which can be placed in a tubular chamber 102 for forming the second chamber of a mouthpiece as hereinbefore described. The lines shown before and after the substrate 100 represent a slower flow of air (in the illustrated example, up to around 0.4 m/s) whereas the flow of air passing through the substrate was found to be faster, between 0.5-1.0 m/s, due to the presence of channels formed within the substrate.

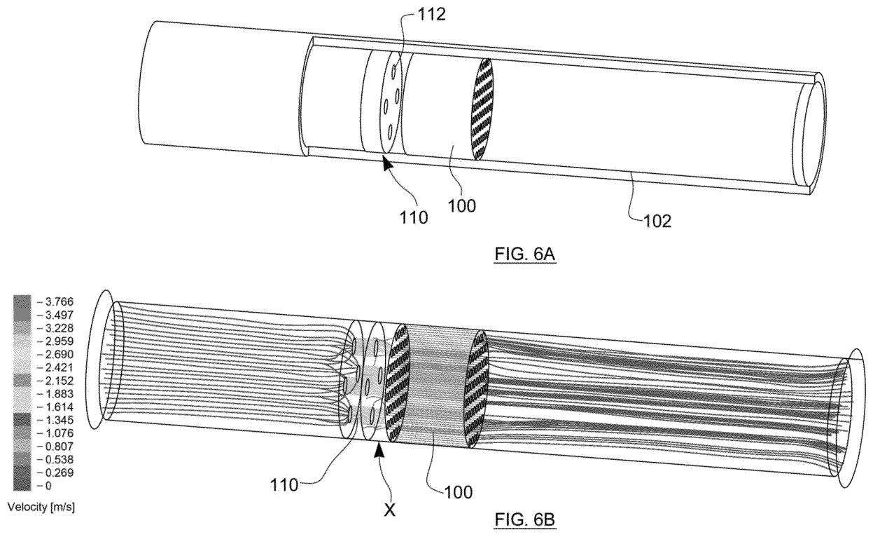

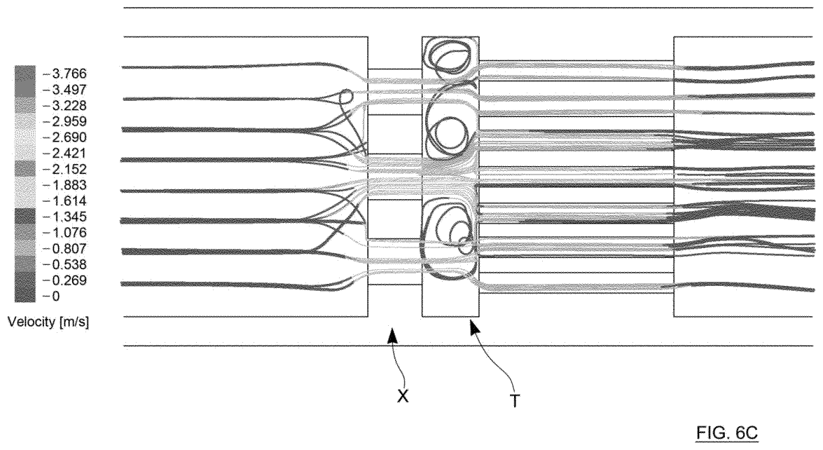

One embodiment of the present invention has a second chamber that is configured to increases air particle velocity into the substrate 100 to create turbulence on entry to the substrate and/or present air to a greater surface area of the substrate. Referring to FIGS. 6A to 6C of the accompanying drawings, a disc 110 with a series of bore holes 112 is provided upstream of the flavour block 100. This serves to increase the velocity of the air flow on entry to the disc 110 (see X in FIGS. 6B and 6C) to above 2 m/s and creates turbulence T (see FIG. 6C) in the air as it exits the disc and enters the substrate 100, thereby slowing down air flow to around 0.5 m/s through the substrate to enhance flavour uptake and increase suction resistance.

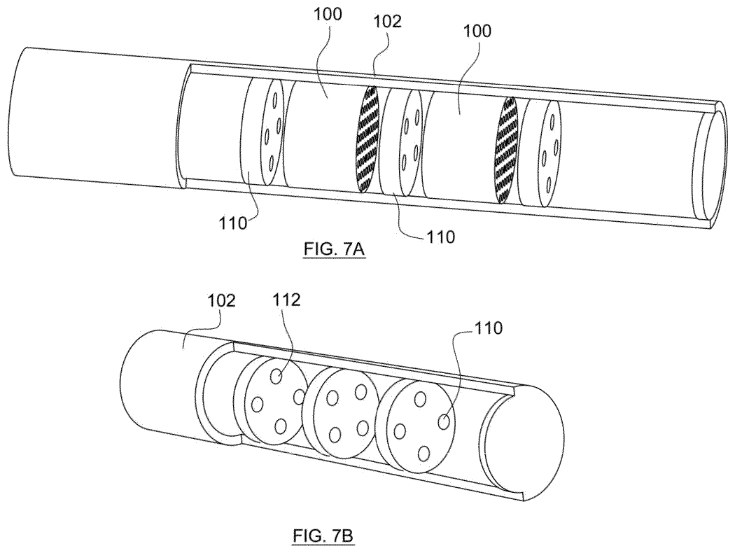

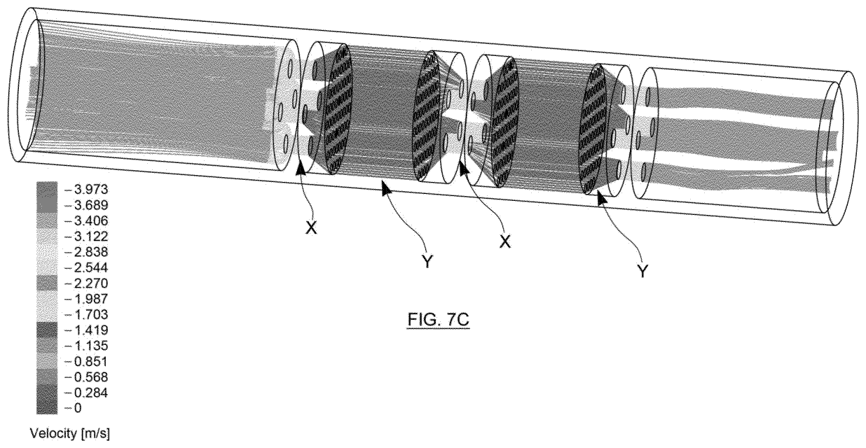

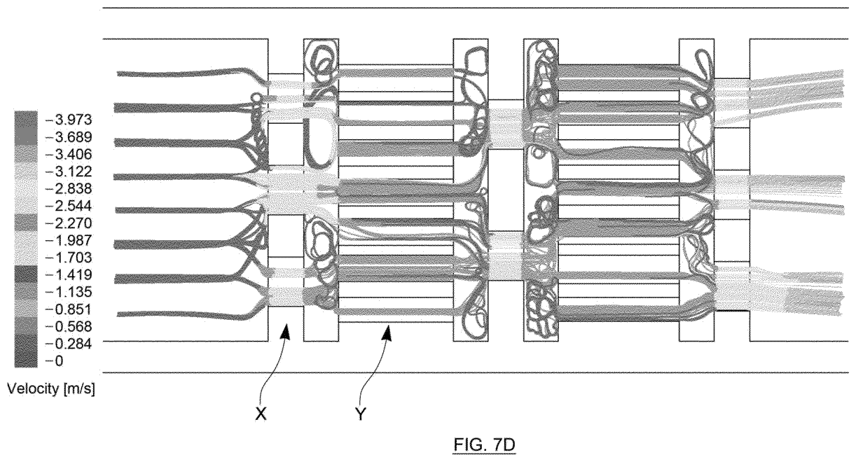

The flavour uptake and suction resistance may be increased further by the incorporation of multiple discs 110 and substrates 100, as illustrated in FIGS. 7A to 7D. Identical features already discussed in relation to FIGS. 6A to 6C are given the same reference numerals. A couple of substrates 100 (not shown in FIG. 7B) containing flavours are provided within the chamber 102 and a disc 110 is provided at an end of each substrate and between the substrates. Again this serves to increase the air velocity at X prior to entry to each substrate, creating turbulence and reducing air flow Y within the substrates. Ideally, the jets of air flow that are created by the bore holes 112 within each disc 110 are provided out of phase (see FIG. 7B) thereby providing a more chaotic air flow to further enhance resistance to air flow through the substrate 100.

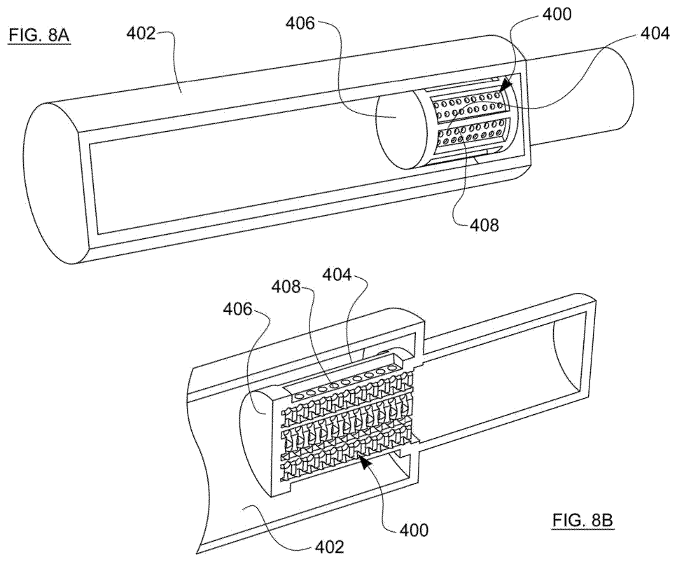

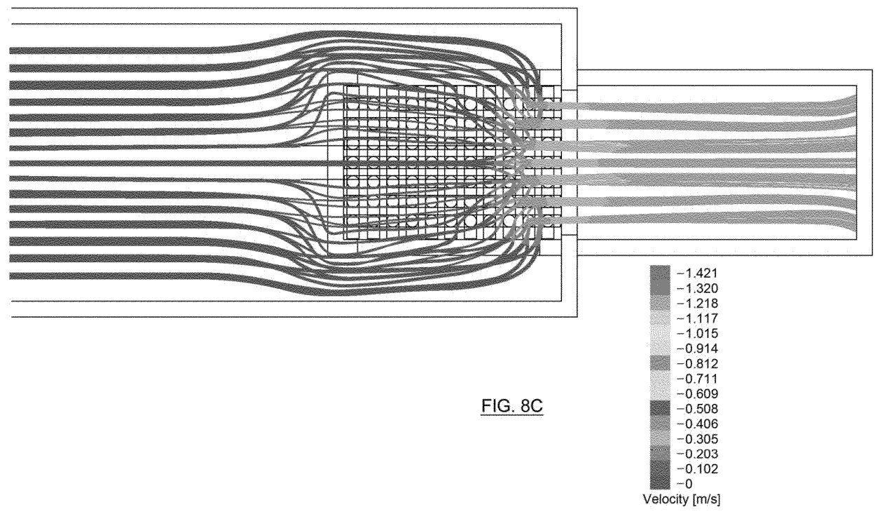

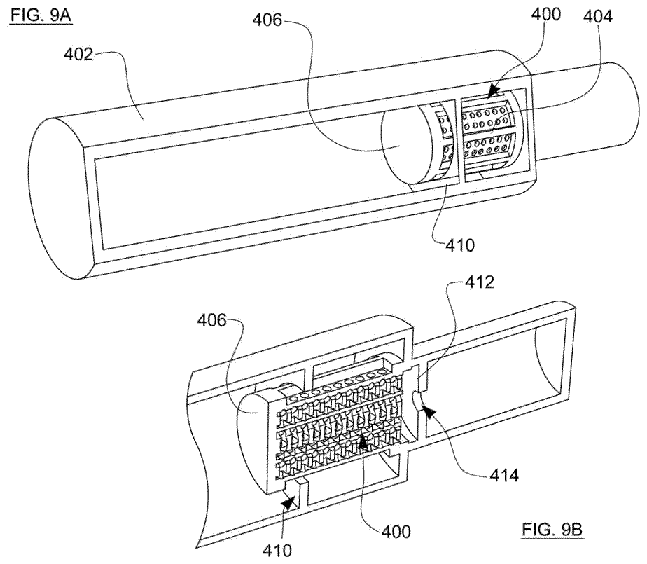

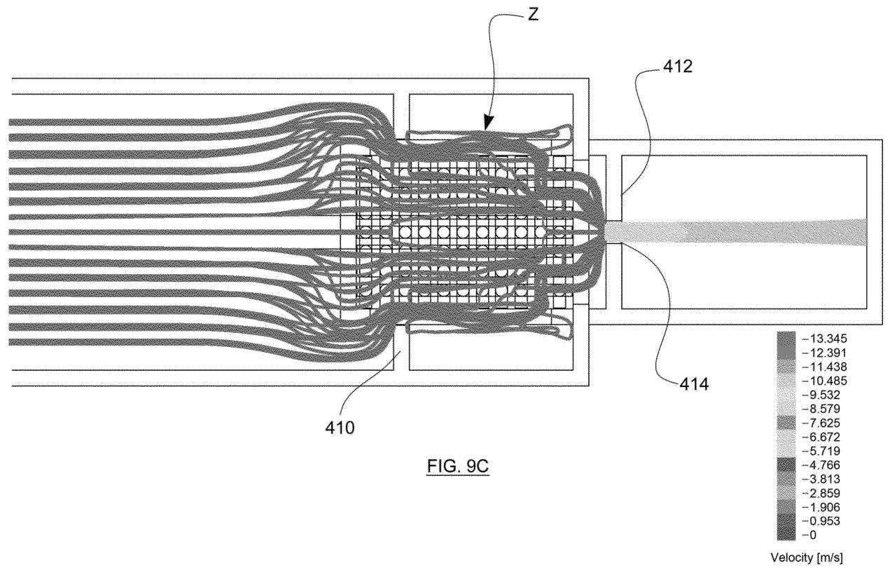

In an alternative embodiment of the present invention, the second chamber of the mouthpiece is configured to direct air flow through as much of the substrate 100 as possible, thereby increasing flavour uptake and suction resistance. FIGS. 8A to 8C and FIGS. 9A to 9C show two embodiments of a chamber 402 which maximizes the surface area of the substrate that comes into contact with air flow through the unit. In FIGS. 8A to 8C, the flavour substrate 400 is housed within a cylindrical cage 404, the cage having a cap 406 at the upstream end of the device and being open at the downstream end. The sides of the cage are provided with multiple spaced apart apertures 408 which serve as entry points for air flow into the substrate. In this manner, air overshoots the initial part of the substrate due to the cap 406 and enters via the multiple inlet apertures 408, thereby directing air through as much of the substrate as possible, as shown in FIG. 8C.

This embodiment may be modified further to increase flavour absorption from the substrate 400 and enhance suction resistance, as shown in FIGS. 9A to 9C. Adding a baffle or vane 410 near to the start of the substrate may further increase air flow into the substrate. Additionally (or alternatively), a downstream end cap 412 with a central aperture 414 may be provided at the outlet end of the substrate 400 to further pull air from its centre. This embodiment provides for a more even and concentrated flow of air throughout the whole surface area of the substrate, as indicated by arrow Z in FIG. 9C.

It is to be appreciated that the mouthpiece or pulmonary delivery device may be provided with a combination of the aforementioned features, such as both a cage and disc combination, to enhance flavour absorption and/or suction resistance.

Alternatively, or additionally, the second chamber of the mouthpiece that contains the flavour block may be provided with at least one subsidiary loop having a confluence with the second chamber before and after the substrate/block to enable air to be recycled through the substrate, thereby enhancing air saturation with flavour and increasing suction resistance. In its most basic form (not shown), one or more side or subsidiary conduits of a narrower cross-sectional diameter than the chamber extend from a junction off the chamber prior to the substrate to a junction beyond the substrate. This enables a proportion of the air that has travelled through the substrate to be diverted back along the subsidiary channel and be re-cycled through the substrate.

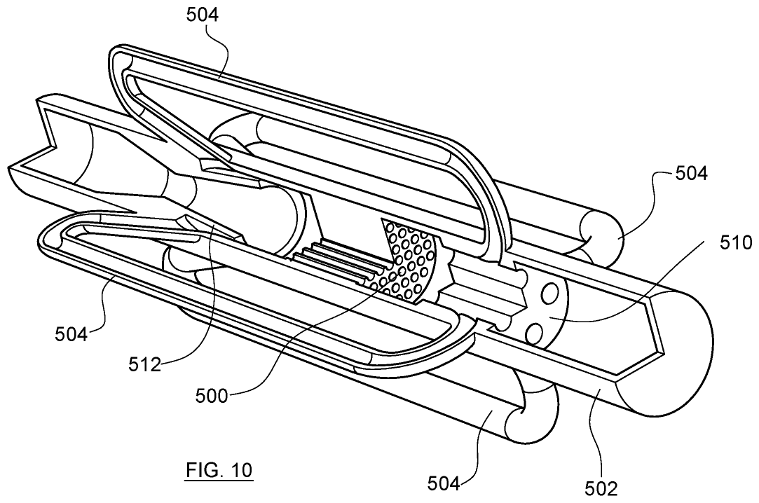

FIG. 10 of the accompanying drawings illustrates one embodiment of a second chamber 502 provided with such a recycling stream. Four subsidiary channels 504 are provided extending from the chamber at a junction prior to the substrate 500 to a junction beyond the substrate. In the illustrated embodiment, a disc 510 with apertures is provided in the chamber at the point where the downstream junctions meet the chamber and a funnel and duct 512 is provided at the point where the upstream junctions meet the chamber. These features aid recycling of the air through the subsidiary channels by encouraging air flow through the downstream inlets to the subsidiary channels while obstructing flow from the chamber 502 into the upstream inlets to the subsidiary channels. Otherwise, air entering into the chamber 502 may enter the upstream inlets and bypass the substrate 500 completely.

Thus, it is important to ensure that the configuration of the chamber and subsidiary conduit(s) is such as to recycle the air back through the substrate rather than cause air to bypass the substrate which would cause the air to have less, rather than more, flavour in the air. This may also be achieved or enhanced by optimizing the cross-sectional area of the subsidiary channels relative to the cross-sectional area of the outlet of the chamber to ensure air velocity is sufficient to prevent bypassing of the chamber.

It is to be appreciated that any of the embodiments discussed could be incorporated into a second chamber of a pulmonary delivery device, preferably being in the form of a mouthpiece having first and second chambers that is connectable to a main body portion of a pulmonary delivery device having a heating element as hereinbefore described. This enables the mouthpiece to be easily replaced or cleaned and allows easy removal of the flavour block and replacement with a new flavor block inserted. Thus, this provides an easy mechanism for a user to change the flavor or their device, for example from cherry to menthol.

The device of the present invention enhances flavour delivery to the user. Any desired flavour may be incorporated into the chamber. A flavour may be provided that mimics a particular food or drink, such as sweet or savoury or even the flavour of a particular meal or dessert. Such flavours would be particularly appropriate for use in a device for the delivery of flavours during enteral feeding wherein food is delivered directly to a patient's stomach via a tube and thus the patient does not benefit from the flavour of the food. Use of a device of the present invention would provide the patient with enhanced satisfaction during feeding due to flavour being delivered to the mouth, activating their taste buds and improving digestion.

In one embodiment, the device or mouthpiece may be provided with a heat source in one or both chambers, preferably wherein the heat source may be switched on or off. More preferably, the main first chamber has a heat source which may be activated as desired. In this manner, the heat source may be switched on if the flavour is mimicking food or drink that is normally served hot but be switched off if the flavour is one of food or drink normally served cold.

Further modifications to a pulmonary delivery device and a mouthpiece for such a device may be made without departing from the principles embodied in the examples described and illustrated herein.

1. A pulmonary delivery device comprising: a first chamber adapted to vaporise a quantity of a first fluid to form a first vapour; a second chamber adapted to atomize a quantity of a second fluid to form a second vapour, and an outlet via which, in use, a user can inhale a mixture of the first and second vapours wherein the second chamber comprises a passive atomiser wherein the second chamber is selectively or continuously in fluid communication with air, the second chamber including a substrate having at least one flavouring or aroma wherein the flavour/aroma is inhaled by drawing air through the second chamber, the second chamber further comprising at least one of an air flow obstruction member and an air flow diversion member to increase uptake of the flavouring or aroma during inhalation of the air.

2. The device as claimed in claim 1, wherein the device includes at least one air flow obstruction member provided upstream of the substrate in the second chamber.

3. The device as claimed in claim 1, wherein the at least one air flow obstruction member comprises a baffle extending at least partially across the chamber, the baffle having at least one air passage therethrough in the form of a hole.

4. (canceled)

5. (canceled)

6. The device as claimed in claim 1, wherein multiple air flow obstruction members are provided in the chamber to adjust air flow through the substrate.

7. The device as claimed in claim 6, wherein multiple discs with bore holes are provided in the chamber, wherein the bore holes of adjacent discs are provided out of phase.

8. The device as claimed in claim 6, wherein the chamber is provided with multiple substrates sandwiched between the air flow obstruction members.

9. (canceled)

10. The device as claimed in claim 1, wherein the substrate is housed within a cage suspended within the chamber, the cage having an end cap at an upstream end of the device and having an outlet at a downstream end.

11. The device as claimed in claim 10, wherein the cage has at least one side provided with multiple spaced apart apertures which serve as entry points for air flow into the substrate.

12. The device as claimed in claim 10 further comprising at least one of a baffle or vane provided near to the start of the substrate, downstream of the end cap and a downstream end cap with at least one aperture provided at the outlet end of the substrate.

13. The device as claimed in claim 10, further comprising a downstream end cap with at least one aperture provided at the outlet end of the substrate, wherein the at least one aperture is provided centrally in the end cap.

14. (canceled)

15. The device as claimed in claim 1, wherein the device includes at least one air diversion member comprising at least one subsidiary channel extending around the part of the chamber that houses the substrate to enable air to be recycled through the substrate, thereby enhancing air saturation with flavour and increasing suction resistance.

16. The device as claimed in claim 15, wherein the at least one subsidiary channel is of a narrower cross-sectional diameter than the chamber and extends from a junction off the chamber prior to the substrate to a junction beyond the substrate, enabling a proportion of the air that has travelled through the substrate to be diverted back along the subsidiary channel and be re-cycled through the substrate.

17. The device as claimed in claim 15, wherein multiple subsidiary channels are provided extending from the chamber at a junction prior to the substrate to a junction beyond the substrate, wherein the cross-sectional area of the at least one subsidiary channel relative to the cross-sectional area of the outlet of the chamber is optimized to ensure air velocity is sufficient to prevent bypassing of the chamber.

18. (canceled)

19. The device as claimed in claim 15 wherein at least one of an entry to or exit from the at least one subsidiary channel is provided with at least one air flow obstruction and/or diversion member to encourage air flow through the downstream inlets to the subsidiary channels while obstructing flow from the chamber into the upstream inlets to the subsidiary channels.

20. The device as claimed in claim 19, wherein at least one disc with at least one aperture is provided in the chamber at a location where a downstream junction meets the chamber and a funnel and duct is provided at a location where an upstream junction meets the chamber, whereby the disc aids the diversion of flow into the subsidiary channel while the funnel and duct obstructs flow into the channels, directing flow through the substrate.

21. The device as claimed in claim 1, wherein at least one chamber is provided with a heat source.

22. A mouthpiece for a pulmonary delivery apparatus, the mouthpiece having a first inlet end and a second outlet end and comprising: a first mouthpiece chamber adapted for receipt within a main body of a pulmonary delivery device at the inlet end of the mouthpiece and a second mouthpiece chamber, the second chamber being adapted for receipt of at least one substrate carrying at least one flavouring or aroma, the second chamber having at least one air inlet at the inlet end whereby the second chamber is selectively or continuously in fluid communication with air to enable inhalation of the flavour/aroma by drawing air through the second chamber, the second chamber further comprising at least one of an air flow obstruction member and an air flow diversion member to increase uptake of the flavouring or aroma during inhalation of the air.

23. The mouthpiece as claimed in claim 22, wherein the mouthpiece includes at least one air flow obstruction member provided upstream of the substrate in the second chamber.

24. (canceled)

25. The mouthpiece as claimed in claim 22, wherein the mouthpiece includes at least one air diversion member comprising at least one subsidiary channel extending around the part of the chamber that houses the substrate to enable air to be recycled through the substrate, thereby enhancing air saturation with flavour and increasing suction resistance.

26. The mouthpiece as claimed in claim 25, wherein the at least one subsidiary channel is of a narrower cross-sectional diameter than the chamber and extends from a junction off the chamber prior to the substrate to a junction beyond the substrate, enabling a proportion of the air that has travelled through the substrate to be diverted back along the subsidiary channel and be re-cycled through the substrate.
